(19) United States
(12) Patent Application Publication
(10) Pub. No.: US 2019/0167921 A1
(43) Pub. Date: Jun. 6, 2019

(54) Status Monitoring Device for Medical Devices

(71) Applicant: Janice Pinckney, Upper Marlboro, MD (US)

(72) Inventor: Janice Pinckney, Upper Marlboro, MD (US)

(21) Appl. No.: 16/201,927

(22) Filed: Nov. 27, 2018

Related U.S. Application Data
(60) Provisional application No. 62/591,020, filed on Nov. 27, 2017.

(51) Int. Cl. A61M 5/50; A61B 5/15; A61M 25/00; A61M 39/02

(52) U.S. Cl. CPC A61M 5/5086; A61B 5/15003; A61B 5/150824; A61M 25/0097; A61M 39/0247; A61B 5/150992; A61M 2005/1588; A61M 2039/0202; A61M 2039/0205; A61M 2039/0258; A61M 2205/3334; A61M 2039/027; A61M 2205/587; A61M 2205/584

(57) ABSTRACT

Provided is a status monitoring device that includes a one-way valve including a change ring and an activator stem. The change ring is configured to change colors (e.g., red, yellow, green or orange) depending on the status of the medical device in operation. The activator stem attached to an interior opening of the one-way valve and is disposed in a vertical direction to interface with a flow channel within the medical device and come into contact with fluids passing therethrough in order to determine whether the medical device is inserted into and operating properly within a patient's body part (e.g., neck, chest, arms and feet).

### I. CROSS-RELATED REFERENCE

This application is a nonprovisional application claiming priority to U.S. Provisional Application Ser. No. 62/591,020 entitled “Status Monitoring Device for Medical Devices”, filed on Nov. 27, 2017, and its contents in its entirety.

### II. TECHNICAL FIELD

The technical field relates generally to a status monitoring device for medical devices (e.g., IV or catheter devices). In particularly, the present invention relates to a status monitoring device for medical devices, capable of monitoring whether the medical device is inserted and operating properly.

### III. BACKGROUND

Today, medical devices (e.g., IV devices) are used to supply or extract fluids to and from patients. It is imperative that these devices are properly inserted and operating in a sufficient manner. However, it may be difficult to assess whether these devices are inserted properly and operating as needed, by only physically viewing by the medical staff. This creates several problems with the use of the medical devices, including for example, causing physical injury or harm to the patients and not providing or extracting the necessary fluids to or from the patients as needed.

### IV. SUMMARY OF EMBODIMENTS

The present invention provides a status monitoring device to be implemented with use of a medical device, capable of continuously monitoring the status of the medical device in real-time.

According to one or more embodiments, a status monitoring device to be implemented with a medical device is provided. The status monitoring device comprises a one-way valve including a change ring and an activator stem. The change ring is configured to change colors (e.g., red, yellow, green or orange) depending on the status of the medical device in operation. The activator stem attached to an interior opening of the one-way valve and is disposed in a vertical direction to interface with a flow channel within the medical device and come into contact with fluids passing therethrough in order to determine whether the medical device is inserted into and operating properly within a patient's body part (e.g., neck, chest, arm, groin, and feet).

The foregoing has broadly outlined some of the aspects and features of various embodiments, which should be construed to be merely illustrative of various potential applications of the disclosure. Other beneficial results can be obtained by applying the disclosed information in a different manner or by combining various aspects of the disclosed embodiments. Accordingly, other aspects and a more comprehensive understanding may be obtained by referring to the detailed description of the exemplary embodiments taken in conjunction with the accompanying drawings, in addition to the scope defined by the claims.

### V. DESCRIPTION OF THE DRAWINGS

FIGS. 1A, 1B and 1C respectively illustrate a bottom view, a perspective view and a side view of a status monitoring device to be implemented within a medical device, according to one or more embodiments of the present invention.

FIG. 2 illustrates the status monitoring device of FIG. 1 connected with a medical device according to one or more embodiments of the present invention.

FIG. 3 illustrates the status monitoring device as shown in FIGS. 1 and 2 and the change ring color indicators associated with different possible statuses of the medical device to be monitored.

FIG. 4 illustrates a block diagram of the status monitoring device as shown in FIGS. 1A through 3, that can be implemented within one or more embodiments of the present invention.

The drawings are only for purposes of illustrating preferred embodiments and are not to be construed as limiting the disclosure. Given the following enabling description of the drawings, the novel aspects of the present disclosure should become evident to a person of ordinary skill in the art. This detailed description uses numerical and letter designations to refer to features in the drawings. Like or similar designations in the drawings and description have been used to refer to like or similar parts of embodiments of the invention.

### VI. DETAILED DESCRIPTION OF THE EMBODIMENTS

As required, detailed embodiments are disclosed herein. It must be understood that the disclosed embodiments are merely exemplary of various and alternative forms. As used herein, the word “exemplary” is used expansively to refer to embodiments that serve as illustrations, specimens, models, or patterns. The figures are not necessarily to scale and some features may be exaggerated or minimized to show details of particular components. In other instances, well-known components, systems, materials, or methods that are known to those having ordinary skill in the art have not been described in detail in order to avoid obscuring the present disclosure. Therefore, specific structural and functional details disclosed herein are not to be interpreted as limiting, but merely as a basis for the claims and as a representative basis for teaching one skilled in the art.

Exemplary embodiments of the present invention provides a status monitoring device to be implemented with a medical device is provided. The status monitoring device is designed to be user-friendly for health care professionals who are inserting intravenous catheters or rendering IV therapy. The device is used to assist in verifying patency and identifying an existing or potential problem or infection at the IV site or within the IV line.

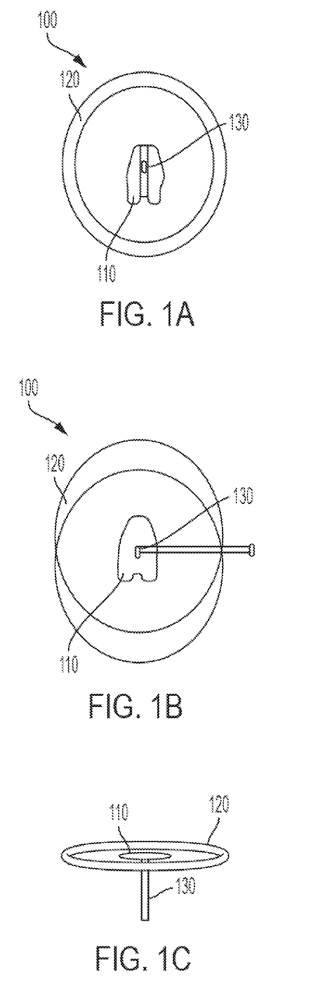

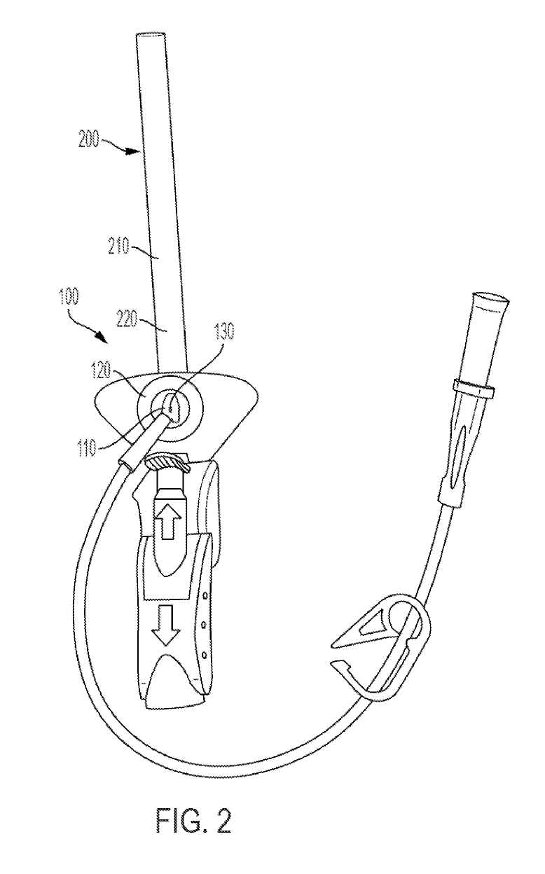

As shown in FIGS. 1A through 4, the status monitoring device 100 comprises a lightweight, one-way valve 110 including a change ring 120 and an activator stem 130. The status monitoring device 100 can be placed on a top surface of a medical device 200 (as depicted in FIG. 2). The medical device 200 can be any existing intravenous device, catheter or port activator device. The one-way valve 110 is disposed above a flow channel 210 within the medical device 200 by which fluids travel through to and from the patient's body part (e.g., neck, chest, arm, groin, and feet). A needle 220 is also included within the flow channel 210 to draw blood from the patient and/or pass fluids through the patient's body.

The status monitoring device 100 may be manufactured to correspond to the clear portion of the medical device 200 (e.g., the catheter) which allows the health care professional to view the fluid (e.g., blood) flowing therethrough the flow channel 210.

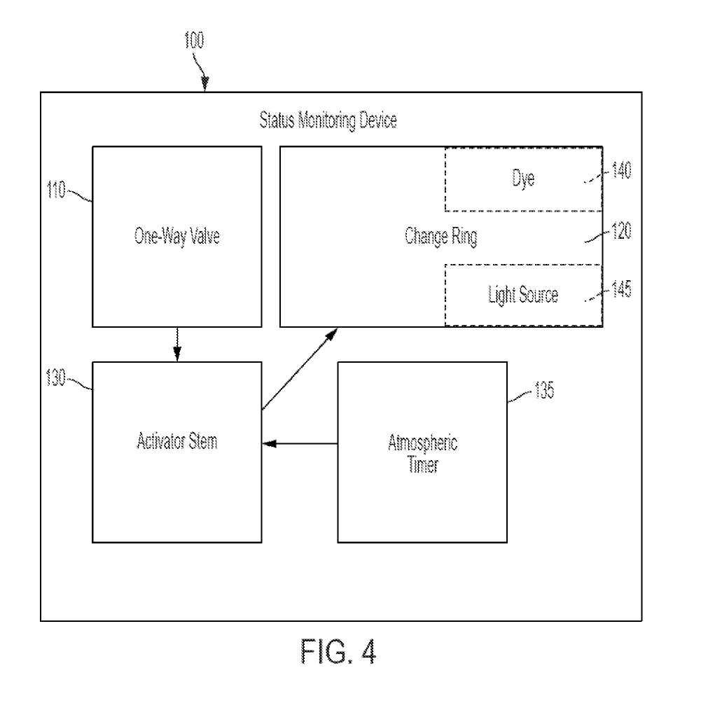

As shown in FIGS. 1B and 1C, the activator stem 130 is disposed in a vertical direction extending away from the one-way valve 110, and within a center region of the flow channel 210 along the y-axis. The activator stem 130 is configured to be activated within approximately 3-7 seconds via an atmospheric timer 135 (as depicted in FIG. 4) for example, to detect a status of the flow of fluid through the flow channel 210 of the medical device 200. The activation can be initiated using any other suitable timing, activation device or operation. The activator stem detects for example, whether the medical device 200 is inserted into and operating properly within a patient's body part (e.g., arm).

An unattached end of the activator stem 130 as shown in FIG. 1C, is disposed to come into contact with the fluid (e.g., certain elements of blood, dermis, and carbon dioxide which can affect the catheter and cause a negative reaction) flowing through the flow channel 210. The opposite end of the activator stem 130 is connected to the one-way valve 110 (e.g., an interior opening thereof) and/or the change ring 120 (as depicted in FIGS. 1A through 1C) such that based on the detection of the activator stem 130, the status of the medical device 200 can be determined. The status monitoring device 100 is mounted on top of any existing medical device 200 with an entry opening for the activator stem 130 to come into contact with. The one-way valve 110, the change ring 120 and the activator stem 130 are not limited to be positioned on top of the medical device 200 and can be positioned in any other suitable location to accomplish the purposes set forth herein. Additional details regarding the detection process are discussed below.

The change ring 130 is configured to change colors (e.g., red, yellow, green or orange) depending on the status of the medical device 200 in operation. Upon initiating the change ring 130 is clear in color. The change ring 120 comprises an opening including an illuminating hypoallergenic non-toxic dye 140 to be dispersed therein. When the activator stem 130 comes in contact with the fluids (e.g., proteins or enzymes), the dye 140 will change color based on the status of the fluid flow within the flow channel 210 or the status of the flow channel 210 and stay until further intervention or a change occurs in the status. The one-way valve prevents the dye from coming into contact with the patients, healthcare professionals or other users. The change ring 130 can be formed of a rubber or light weight plastic material or any other material suitable for the purpose herein. Alternative to the use of dye 140, a light source 145 (as depicted in FIG. 4) can be provided within the change ring 130 in order to facilitate the change in color based on the status of the medical device 200. Other suitable illumination sources may be used herein to facilitate the color illumination as desired.

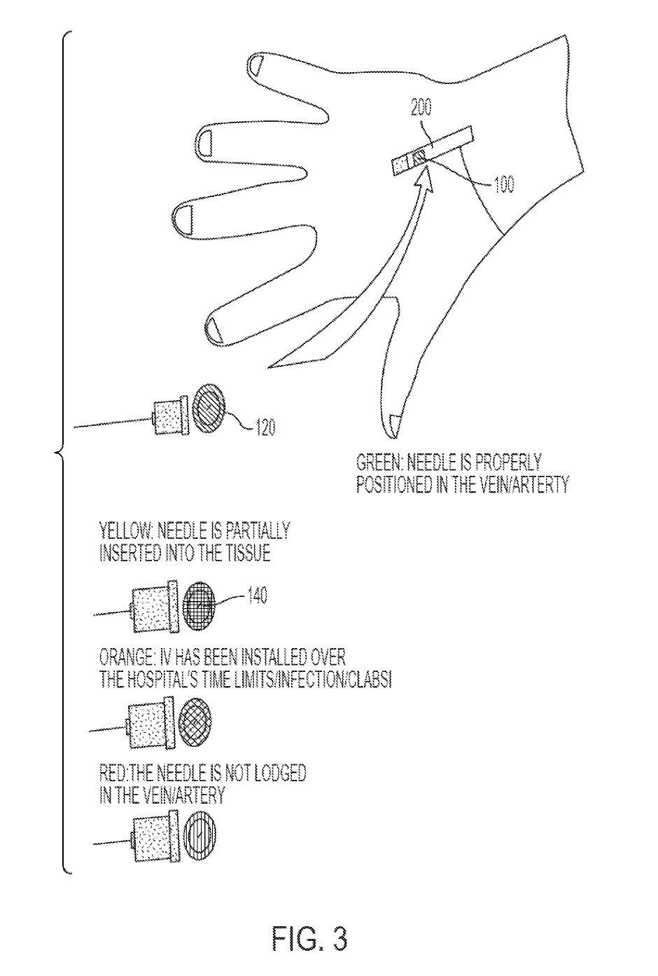

As shown in FIG. 3, the change ring 120 of the status monitoring device 100 indicates a status of the medical device 200 by use of a color (e.g., a dye). If the color is green, the activator stem 120 has come into direct contact with plasma and red blood cells, for example. The change ring 120 will remain green during patency of the IV, signaling safe use and proper positioning within the vein, for example.

If the color is yellow, this is an indication that the medical device 200 (e.g., the IV) has been infiltrated which means that it is no longer in the vein but instead in the dermis. Therefore, the medical device 200 needs to be removed and replaced. The status monitoring device 100 will stay yellow in color until removal of the medical device 200. It can start out green in color and then turn yellow once the medical device 200 has migrated into the dermis rendering it no longer patent.

The change ring 120 is red in color immediately after 3-7 seconds when the medical device 200 (e.g., the IV) does not enter into a vein and instead is in the dermis. The medical device 200 is therefore not safe to use and the status monitoring device 100 remains red until the medical device 200 is removed.

The change ring 120 is orange in color when the medical device 200 (e.g., a catheter) encounters any toxins or bacteria in the vein. The status monitoring device 100 will detect toxins and bacteria in catheters such as central venous catheters (“CVC”) inserted into large veins (e.g., internal jugular, right atrial line, ports, and peripherally inserted central catheter lines, etc.) The status monitoring device 100 will remain orange in color when used peripherally after approximately 3-7 days has passed indicating the intravenous site and/or dressing needs to be changed to allow health care professionals to be proactive in preventing bacterial infections.

The status monitoring device 100 can be attached or mounted to any intravenous catheter or butterfly needle device or other existing IV device. The one-way way valve 110 gets activated when the activator stem 130 encounters blood or body fluids and carbon dioxide, and remains activated for approximately 3-7 days. The status monitoring device illuminates at night making it more visible.

There are several advantages associated with the status monitoring device 100 of the present invention. For example, the device 100 makes it easier to prevent and identify IV infiltrations, extravasations, and line sepsis. It reduces incidences of facility acquired infections, CLABSI (central line-associated bloodstream infection), and infections caused by low frequency of IV dressing changes. There will be no reading or writing on the IV sites. In some cases the label is often illegible to read either the date, signature, or initial or it has been removed. This device 100 reduces time and cost for facilities and health care providers, and is proactive in preventing injuries to patients. The healthcare providers can recognize the status of an IV at a glance.

This written description uses examples to disclose the invention, including the best mode, and also to enable any person skilled in the art to practice the invention, including manufacturing operations. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. A status monitoring device to be implemented with a medical device, and comprising:
a one-way valve comprising:
a change ring configured to change colors depending on the status of the medical device in operation; and
an activator stem attached to an interior opening of the one-way valve and disposed in a vertical direction to interface with a flow channel within the medical device and come into contact with fluids passing therethrough in order to determine whether the medical device is inserted into and operating properly within a patient's body part.

2. The status monitoring device of claim 1, wherein the status monitoring device is a lightweight device, to be placed on a top surface of the medical device.

3. The status monitoring device of claim 2, wherein the medical device is one of an intravenous device, a catheter or a port activator device.

4. The status monitoring device of claim 3, wherein the one-way valve is disposed above the flow channel within the medical device by which fluids travel through to and from the patient's body part.

5. The status monitoring device of claim 4, wherein a needle is disposed within the flow channel to draw fluid from a patient and/or pass fluids through the patient's body part.

6. The status monitoring device of claim 5, wherein the status monitoring device corresponds to a clear portion of the medical device to allow for viewing of any fluid flowing therethrough the flow channel.

7. The status monitoring device of claim 1, wherein the activator stem is disposed in the vertical direction extending away from the one-way valve, and within a center region of the flow channel along a y-axis.

8. The status monitoring device of claim 7, wherein the status monitoring device further comprising: an atmospheric timer, wherein the activator stem is configured to be activated within approximately 3-7 seconds via the atmospheric timer, to detect a status of the flow of fluid through the flow channel of the medical device.

9. The status monitoring device of claim 8, wherein an unattached end of the activator stem is disposed to come into contact with any fluid flowing through the flow channel, and wherein an opposite end of the activator stem is connected to the one-way valve and/or the change ring wherein based on the detection of the activator stem, the status of the medical device can be determined.

10. The status monitoring device of claim 1, wherein upon initiating, the change ring is clear in color, and wherein the change ring comprises an opening including an illuminating hypoallergenic non-toxic dye to be dispersed therein.

11. The status monitoring device of claim 10, wherein when the activator stem comes in contact with any fluids, the dye changes color based on the status of any fluid flow within the flow channel or the status of the flow channel and stay unchanged until further intervention or a change occurs in either status.

12. The status monitoring device of claim 11, wherein the one-way valve is configured to block the dye from coming into contact with the patient's body part.

13. The status monitoring device of claim 1, wherein the change ring is formed of a rubber or light weight plastic material.

14. The status monitoring device of claim 13, wherein the change ring further comprises a light source therein configured to change in color based on the status of the medical device.

15. The status monitoring device of claim 10, wherein if the color is green, then the activator stem has come into direct contact fluids signaling safe use and proper positioning of the medical device.

16. The status monitoring device of claim 10, wherein if the color is yellow, then it is determined that the medical device has been infiltrated is not properly positioned or needs to be replaced, wherein the color remains yellow until removal of the medical device.

17. The status monitoring device of claim 10, wherein if the color is red immediately after 3-7 seconds then the medical device is not safe to use and the status monitoring device remains red until the medical device is removed.

18. The status monitoring device of claim 10, wherein if the color is orange then the medical device 200 is encountering any toxins or bacteria in patient's body part, and the color remains orange when used peripherally after approximately 3-7 days has passed indicative that medical device needs to be replaced.

19. The status monitoring device of claim 1, wherein the status monitoring device is illuminative in a dark environment.
